(19) United States
(12) Patent Application Publication
(10) Pub. No.: US 2021/0294089 A1
(43) Pub. Date: Sep. 23, 2021

(54) IMAGE CAPTURING MODULE AND ENDOSCOPE

(71) Applicant: Fujikura Ltd., Tokyo (JP)

(72) Inventor: Yoshinobu Numasawa, Sakura-shi (JP)

(73) Assignee: Fujikura Ltd., Tokyo (JP)

(21) Appl. No.: 17/254,000

(22) PCT Filed: Jul. 1, 2019
(86) PCT No.: PCT/JP2019/026078

§ 371 Date: Dec. 18, 2020

(30) Foreign Application Priority Data
Jul. 2, 2018 (JP) 2018-125901

(51) Int. Cl. G02B 23/24; G02B 5/02; H04N 5/225

(52) U.S. Cl. CPC G02B 23/2461; G02B 5/0242; G02B 5/0278; H04N 2005/2255; H04N 5/2252; H04N 5/2256; G02B 23/2484

(57) ABSTRACT

An image capturing module includes an image capturing element, a light emitting element, and a case that is configured to hold the image capturing element and the light emitting element. The case has a light projection aperture that is configured to open toward the front surface of the case, and a light housing part that is located behind the light projection aperture to house the light emitting element. The light housing part and the light projection aperture are connected to each other inside the case, and the rear end of the light projection aperture is configured to open toward a light emitting surface of the light emitting element. The opening area of the rear end of the light projection aperture is smaller than the area of the light emitting surface of the light emitting element.

### TECHNICAL FIELD

The present invention relates to an image capturing module and an endoscope. Priority is claimed to Japanese Patent Application No. 2018-125901, filed Jul. 2, 2018, the contents of which are incorporated herein by reference in their entirety.

### BACKGROUND

Image capturing modules used for endoscopes or the like have been known heretofore as shown in the following Patent Document 1. The image capturing modules includes an image capturing element, a light emitting element, and a case that holds these elements. A case is formed with a light housing part that is recessed rearward from a front surface of the case, and the light emitting element is housed in the light housing part.

### PATENT LITERATURE

### Patent Document 1

Japanese Unexamined Patent Application, First Publication No. 2007-252843

When the image capturing module described in Patent Document 1 is assembled, the light emitting element easily falls off toward the front side of the case due to the structure of the image capturing module.

One or more embodiments of the invention provide an image capturing module and an endoscope capable of preventing the light emitting element from falling off.

### SUMMARY

An image capturing module according to one or more embodiments of the present invention includes an image capturing element; a light emitting element; and a case that is configured to hold the image capturing element and the light emitting element. The case has a light projection aperture that is configured to open to a front surface of the case, and a light housing part that is located behind the light projection aperture to house the light emitting element. The light housing part and the light projection aperture are connected to each other inside the case, and a rear end of the light projection aperture is configured to open toward a light emitting surface of the light emitting element. An opening area of the rear end of the light projection aperture is smaller than an area of the light emitting surface of the light emitting element.

In the above embodiments, the light projection aperture is connected to the light housing part, and the rear end of the light projection aperture is configured to open toward the light emitting surface of the light emitting element. For this reason, the light emitted from the light emitting element can be emitted to the front of the image capturing module through the light projection aperture.

Also, since the opening area of the rear end of the light projection aperture is smaller than the area of the light emitting surface of the light emitting element, it is possible to inhibit the light emitting element from falling off to the front of the image capturing module through the light projection aperture. Moreover, since the opening area of the rear end of the light projection aperture is smaller than the area of the light emitting surface of the light emitting element, the light emitting element can be easily positioned.

The light projection aperture may have a tapered part that is inclined such that a cross-sectional area of the light projection aperture increases toward a front opening edge of the light projection aperture.

In this case, the light emitted from the light emitting element is radiated to the front of the image capturing module while spreading along the tapered part. For this reason, it is possible to illuminate the image capturing target in a wider range than the opening in front of the light projection aperture.

The tapered part may be formed from a rear opening edge of the light projection aperture to the front opening edge thereof.

In this case, the light emitted from the light emitting element is more directly emitted forward along the tapered part. Therefore, it is possible to more efficiently illuminate the image capturing target.

A transparent resin may be filled inside the light projection aperture.

In this case, it is possible to inhibit dirt and the like from accumulating inside the light projection aperture.

The transparent resin may contain light diffusing particles.

In this case, the light emitted from the light emitting element is diffused by the light diffusing particles when passing through the light projection aperture. Therefore, it is possible to illuminate the image capturing target in a wider range.

An area A1 of the light emitting surface and an opening area A2 of the front end of the light projection aperture may satisfy A1≤A2.

In this case, light can be emitted through the opening in front of the light projection aperture having a larger area than the light emitting surface. That is, it is possible to make the ease of positioning the light emitting element and the wide range of the light projection range to the image capturing target compatible with each other.

An endoscope according to one or more embodiments of the present invention may use the above image capturing module.

According to the above embodiments of the present invention, it is possible to provide the image capturing module and the endo scope capable of preventing the light emitting element from falling off.

### BRIEF DESCRIPTION OF DRAWINGS

FIG. 1 is a perspective view of a distal end of an endoscope including an image capturing module of the present embodiments.

FIG. 2 is a view showing a section obtained by partially cutting the image capturing module of FIG. 1.

FIG. 3 is a perspective view showing a connection state between a light emitting element and wiring lines.

FIG. 4 is a sectional view taken along line IV-IV in FIG. 2.

FIG. 5 is a view in which a sectional portion of FIG. 2 is extracted.

FIG. 6 is a perspective view of a case viewed from the rear end side.

FIG. 7 is a sectional view taken along line VII-VII of FIG. 4.

FIG. 8A is a sectional view of an image capturing module according to a modified example of the present embodiments.

FIG. 8B is a sectional view of an image capturing module according to a modified example of the present embodiments.

FIG. 8C is a sectional view of an image capturing module according to a modified example of the present embodiments.

### DETAILED DESCRIPTION

Hereinafter, an image capturing module of the present embodiments and an endoscope including the image capturing module will be described with reference to the drawings.

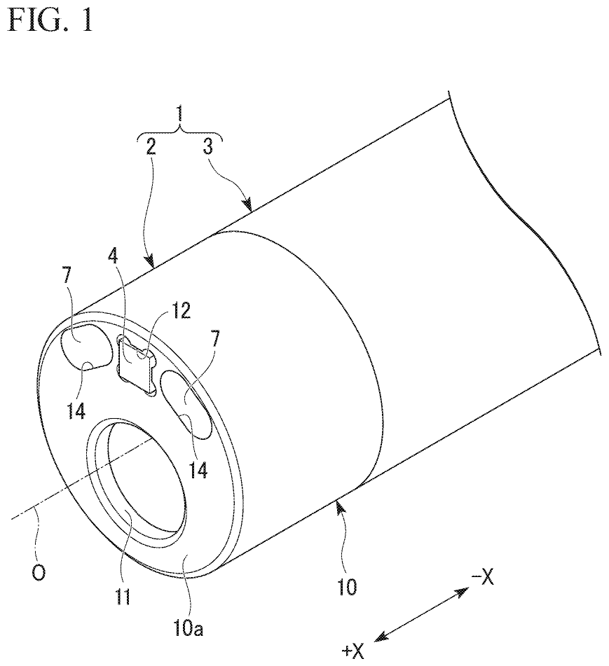

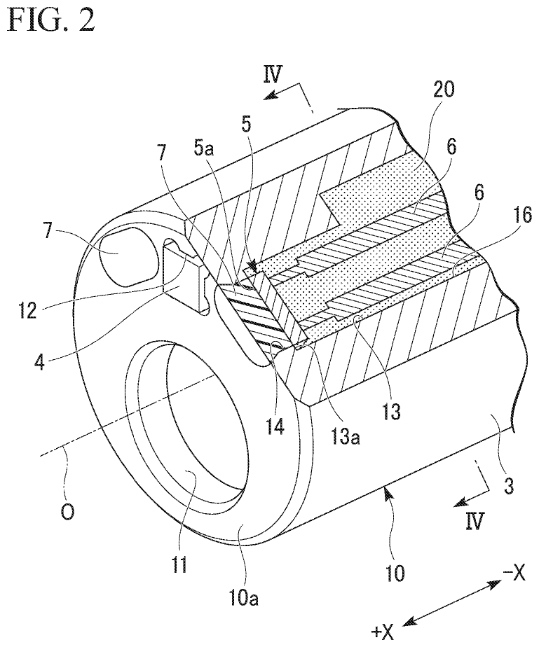

As shown in FIG. 1, the endoscope 1 includes an image capturing module 2 and a tube 3. The image capturing module 2 includes an image capturing element 4, a light emitting element 5 (see FIG. 2), and a case 10 that holds the image capturing element 4 and the light emitting element 5. The case 10 is formed in a substantially columnar shape such that the outer diameter of the case 10 is equivalent to the outer diameter of the tube 3. The case 10 is attached to the tip of the tube 3.

### (Direction Definition)

In the present embodiments, a central axis of the case 10 is referred to as a central axis O. Additionally, a direction along the central axis O is referred to as a longitudinal direction X. In the longitudinal direction X, the case 10 side as viewed from the tube 3 is referred to as a front side (+X side), and the tube 3 side as viewed from the case 10 is referred to as a rear side (−X side). A section orthogonal to the longitudinal direction X is referred to as a cross-section, and the area of the cross-section is referred to as a cross-sectional area.

As shown in FIG. 1, the image capturing module 2 is configured to capture an image of an image capturing target on the front side of the endoscope 1 with the image capturing element 4 while irradiating the front side of the endoscope 1 with the light emitted from the light emitting element 5. Additionally, the case 10 is formed with a hole (channel 11) that penetrates the case 10 in the longitudinal direction X. The channel 11 is connected to the inside of the tube 3. By housing a treatment tool in the channel 11, the endoscope 1 can be used as a catheter. Examples of the treatment tool include various forceps, a snare, a guide wire, a stent, a laser treatment tool, a high-frequency treatment tool, and the like.

Hereinafter, the configuration of the respective parts will be described in more detail.

### (Tube)

As shown in FIG. 1, the tube 3 is formed in a substantially cylindrical shape.

The tube 3 has flexibility. As a material of the tube 3, a material having an insulating property such as silicon, polyurethane, polyethylene, or polytetrafluoroethylene (PTFE) can be used. In addition, a metallic material may be used for the tube 3. Various wiring lines of the image capturing module 2 are inserted through the tube 3.

### (Image Capturing Element)

As shown in FIG. 1, the image capturing element 4 is housed in a camera housing part 12 formed in the case 10. As the image capturing element 4, for example, a complementary metal oxide semiconductor (CMOS) or the like can be used. Wiring lines (not shown) are connected to the image capturing element 4, and the wiring lines are housed in the case 10 and the tube 3.

### (Light Emitting Element)

As shown in FIG. 2, the image capturing module 2 of the present embodiments has two light emitting elements 5 with the image capturing element 4 interposed therebetween. The two light emitting elements 5 are housed in the two light housing parts 13 formed in the case 10, respectively. The configurations of the two light emitting elements 5 are the same. However, the number of light emitting elements 5 included in the image capturing module 2 may be appropriately changed, or the light emitting elements 5 having different configurations may be used in combination.

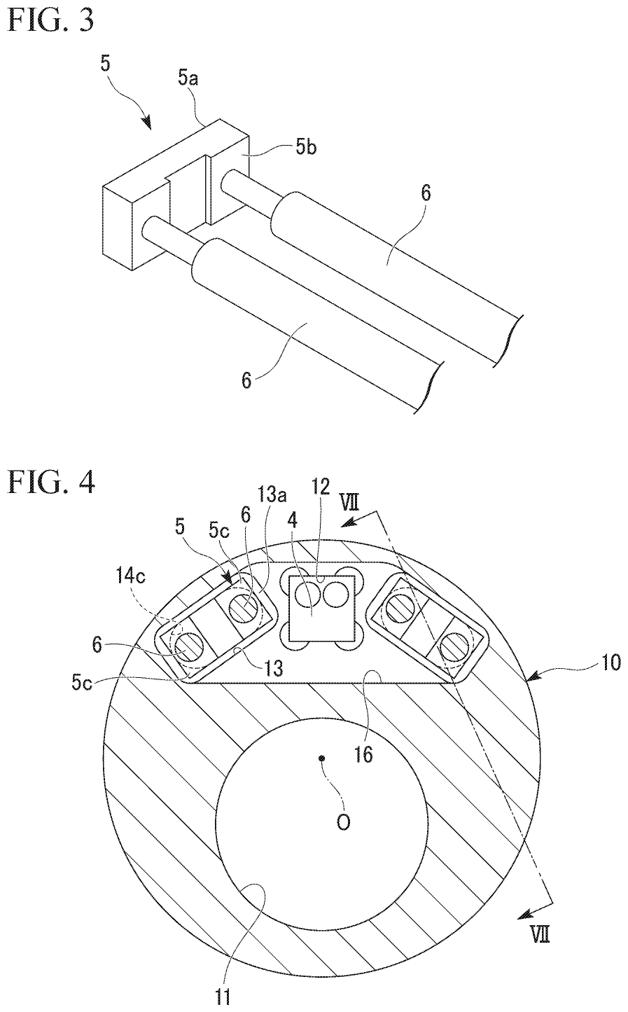

As shown in FIG. 3, as the light emitting element 5, a surface-mounted light emitting diode or the like can be used. The light emitting element 5 of the present embodiments has a flat plate shape with a thickness of about 0.25 mm and is formed in an oblong shape (rectangular shape) when viewed from the front side in the longitudinal direction X. The light emitting element 5 has a light emitting surface 5a that faces forward. Wiring lines 6 are connected to a surface 5b of the light emitting element 5 opposite to the light emitting surface 5a by solder or the like (not shown). Although the number of wiring lines 6 is two in FIG. 3, the number of wiring lines 6 may be appropriately changed. As shown in FIG. 2, the wiring lines 6 are inserted into the tube 3.

### (Case)

As shown in FIG. 1, the case 10 is attached to a front end of the tube 3. A front surface 10a of the case 10 is formed in a substantially circular shape when viewed from the front side in the longitudinal direction X. The case 10 of the present embodiments is formed by cutting a metallic material such as stainless steel. However, the material and the processing method of the case 10 can be appropriately changed.

As shown in FIG. 2, the case 10 is formed with the channel 11, the camera housing part 12, the light housing part 13, and a light projection aperture 14. Two light housing parts 13 and two light projection aperture 14 are formed in accordance with the number of the light emitting elements 5 included in the image capturing module 2. When viewed from the front side in the longitudinal direction X, the two light projection apertures 14 are disposed so as to sandwich the camera housing part 12.

The channel 11 is a through hole formed in a substantially circular shape when viewed from the front side in the longitudinal direction X.

The camera housing part 12 is a through hole formed in a substantially square shape when viewed from the front side in the longitudinal direction X. The image capturing element 4 is fitted inside the camera housing part 12. An escape shape when the case 10 is cut is formed at a corner of the camera housing part 12. An adhesive (not shown) is filled in a gap between the camera housing part 12 and the image capturing element 4. Accordingly, the gap in the camera housing part 12 is buried and the image capturing element 4 is fixed to the case 10.

As shown in FIG. 2, the light housing part 13 is a recess that is recessed from the rear end of the case 10 toward the front. The light housing part 13 is located behind the light projection aperture 14. The light emitting element 5 is housed inside the light housing part 13 with its light emitting surface facing forward. When viewed from the front side in the longitudinal direction X, a gap is provided between an inner surface of the light housing part 13 and the light emitting element 5. An insulating resin 20 is filled in the gap between the inner surface of the light housing part 13 and the light emitting element 5.

As shown in FIG. 4, the light housing part 13 is formed in a substantially oblong shape larger than the light emitting element 5 when viewed from the front side in the longitudinal direction X. In FIG. 4, the illustration of the resin 20 is omitted.

As shown in FIG. 2, the light projection aperture 14 is a recess that is recessed rearward from the front surface 10a of the case 10. The light projection aperture 14 is connected to the light housing part 13 inside the case 10. A rear end of the light projection aperture 14 opens toward the light emitting surface 5a of the light emitting element 5. In other words, the light projection aperture 14 and the light housing part 13 are connected to each other to form a hole that penetrates the case 10 in the longitudinal direction X.

As shown in FIG. 2, a transparent resin 7 is filled inside the light projection aperture 14. As the transparent resin 7, a resin having high light transmittance can be used. For example, acrylic, UV curable resin, or the like is suitable as the transparent resin 7. When the image capturing module 2 is manufactured, the transparent resin 7 is filled and cured inside the light projection aperture 14 after the light emitting element 5 is fixed in the light housing part 13.

The transparent resin 7 may include light diffusing particles having a property of diffusing light. Alumina particles and the like can be used as the light diffusing particles.

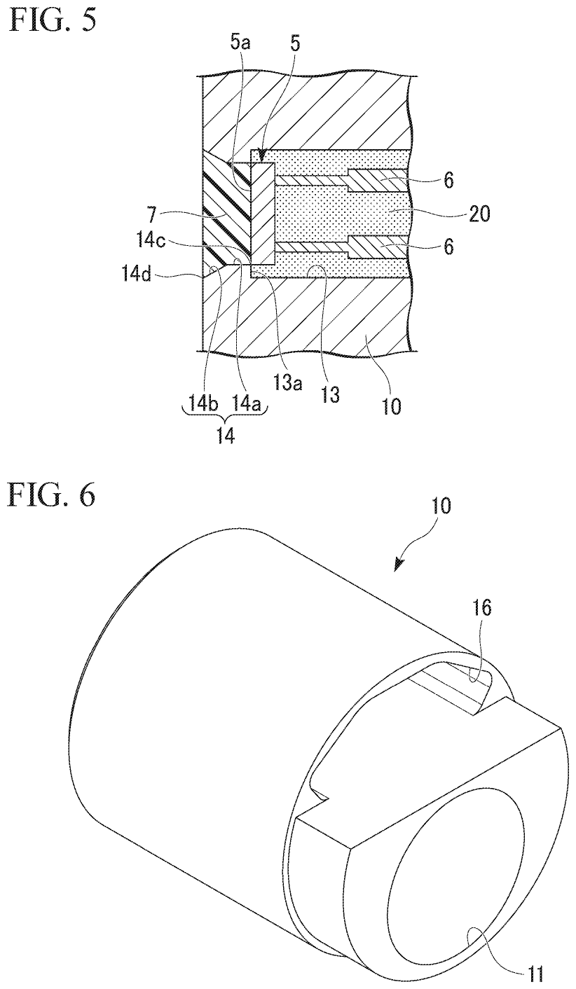

As shown in FIG. 5, the light projection aperture 14 has a straight part 14a and a tapered part 14b. An inner surface of the straight part 14a extends parallel to the longitudinal direction X. At least a portion of the inner surface of the tapered part 14b is inclined with respect to the longitudinal direction X such that the cross-sectional area of the light projection aperture 14 increases toward a front opening edge 14d. Due to this shape, the opening area of a front end of the light projection aperture 14 (the cross-sectional area of the light projection aperture 14 at the front opening edge 14d) is larger than the opening area of the rear end of the light projection aperture 14 (the cross-sectional area of the light projection aperture 14 at a rear opening edge 14c).

Additionally, an area A1 of the light emitting surface 5a and an opening area A2 of the front end of the light projection aperture 14 satisfy A1<A2.

An inner surface of the light projection aperture 14 (inner surfaces of the straight part 14a and the tapered part 14b) may be mirror-finished. Accordingly, since the light emitted from the light emitting element 5 is reflected on the inner surface of the light projection aperture 14 when passing through the light projection aperture 14, attenuation can be suppressed, and the image capturing target can be more efficiently illuminated.

As shown in FIGS. 4 and 6, a rear end of the camera housing part 12 and rear ends of the two light housing parts 13 are connected to a common recess 16. The recess 16 is formed so as to be recessed from the +X direction of the case 10 toward the −X direction. The recess 16 is formed in a substantially trapezoidal shape when viewed from the rear. In a case where the case 10 is cut, by forming the recess 16 and then forming the camera housing part 12 and the light housing part 13, it is possible to shorten the processing time when the camera housing part 12 and the light housing part 13 are formed. In particular, since the camera housing part 12 and the light housing part 13 are parts for positioning the image capturing element 4 and the light emitting element 5, there is a case where relatively high processing accuracy may be required. Even in such a case, by providing the recess 16 as in the present embodiments, the case 10 can be formed with high accuracy in a short processing time.

### (Abutting Surface)

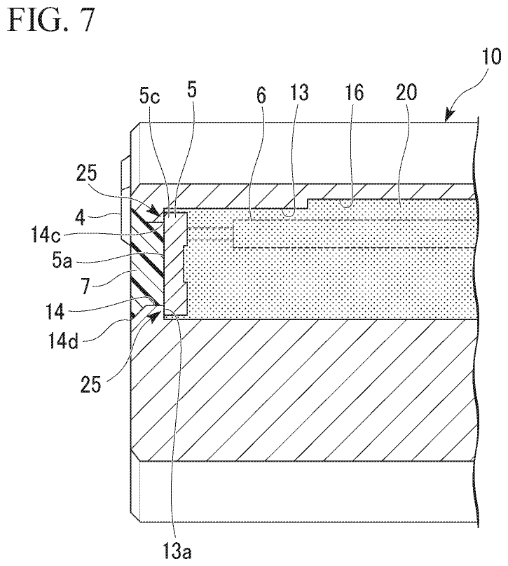

In the present embodiments, as shown in FIG. 7, the light housing part 13 and the light projection aperture 14 are connected to each other inside the case 10, and a step 25 that faces rearward is formed at a connecting portion between the light housing part 13 and the light projection aperture 14. In the present embodiments, the step 25 is used as a abutting surface 13a for butting (contacting) the light emitting surface 5a of the light emitting element 5.

As shown in FIG. 7, the abutting surface 13a is a portion of the inner surface of the light housing part 13. The abutting surface 13a extends from the rear opening edge 14c of the light projection aperture 14 so as to widen outward. The position of the light emitting element 5 in the longitudinal direction X is determined by the contact of the light emitting surface 5a of the light emitting element 5 with the abutting surface 13a. The opening area of the rear end of the light projection aperture 14 (the cross-sectional area of the light projection aperture 14 at the rear opening edge 14c) is smaller than the area of the light emitting surface 5a.

In the present embodiments, as shown in FIG. 4, the rear opening edge 14c of the light projection aperture 14 and the light emitting surface 5a of the light emitting element 5 are both formed in a substantially oblong shape. The long-side dimension of the rear opening edge 14c is equivalent to the long-side dimension of the light emitting surface 5a. The same short-side dimension of the rear opening edge 14c is equivalent to the short-side dimension of the light emitting surface 5a. On the other hand, the rear opening edge 14c of the light projection aperture 14 is formed in a substantially oblong shape with curved corners. Also, the portion of the abutting surface 13a outside the above curve is in contact with the light emitting surface 5a of the light emitting element 5. For this reason, most of the light emitting surface 5a faces the light projection aperture 14, and angular parts 5c of the light emitting surface 5a is in contact with the abutting surface 13a, whereby the position and posture of the light emitting element 5 are determined. In FIG. 5, it seems that the light emitting surface 5a appears not to be in contact with the abutting surface 13a. This is because only the angular parts 5c of the light emitting surface 5a are in contact with the abutting surface 13a as shown in FIG. 4.

### (Operation)

As described above, in the present embodiments, the light projection aperture 14 is connected to the light housing part 13, and the rear end of the light projection aperture 14 opens toward the light emitting surface 5a of the light emitting element 5. For this reason, the light emitted from the light emitting element 5 can be emitted to the front of the image capturing module 2 through the light projection aperture 14.

Also, since the opening area behind the light projection aperture 14 (the area inside the rear opening edge 14c) is structured to be smaller than the area of the light emitting surface 5a, for example, the light emitting element 5 can be inhibited from falling off to the front of the image capturing module 2 through the light projection aperture 14.

Additionally, since the image capturing module 2 is configured such that the opening area behind the light projection aperture 14 (the area inside the rear opening edge 14c) is smaller than the area of the light emitting surface 5a, when the image capturing module 2 is assembled, a procedure of housing the light emitting element 5 in the light housing part 13 from the rear of the case 10 can be adopted. Therefore, the work of inserting the wiring lines 6 of the light emitting element 5 through the light housing part 13 is unnecessary, and the work of fixing the light emitting element 5 in the light housing part 13 is also easily performed. Additionally, since a case where the image capturing element 4, the treatment tool housed in the channel 11, and the like are also attached from the rear of the case 10 is general, these components and the light emitting element 5 can be assembled in the same manner.

In this way, according to the image capturing module 2 of the above embodiments, the manufacturing efficiency of the image capturing module can be improved.

Additionally, since the light projection aperture 14 has the tapered part 14b, the light emitted from the light emitting element 5 is radiated to the front of the image capturing module 2 while spreading along the tapered part 14b. Therefore, it is possible to illuminate the image capturing target in a wider range than the opening in front of the light projection aperture 14.

Additionally, since the transparent resin 7 is filled inside the light projection aperture 14, it is possible to inhibit dirt and the like from accumulating inside the light projection aperture 14. Moreover, since the transparent resin 7 functions as an insulator, the light emitting element 5 and the image capturing target can be more reliably insulated.

Additionally, in a case where the transparent resin 7 contains light diffusing particles, the light emitted from the light emitting element 5 is diffused by the light diffusing particles when passing through the light projection aperture 14. Therefore, it is possible to illuminate the image capturing target in a wider range.

Additionally, the rear opening edge 14c of the light projection aperture 14 is formed in a substantially oblong shape with curved corners. Also, the portion of the abutting surface 13a outside the above curve is in contact with the light emitting surface 5a of the light emitting element 5. Due to this configuration, it is possible to make most other than the angular parts 5c face the light projection aperture 14 while butting the angular parts 5c of the light emitting surface 5a against the abutting surface 13a. Therefore, it is possible to more efficiently radiate the light emitted by the light emitting element 5 forward through the light projection aperture 14 while positioning the light emitting element 5.

In addition, the technical scope of the present invention is not limited to the above-mentioned embodiments, and various changes can be made without departing from the spirit of the present invention.

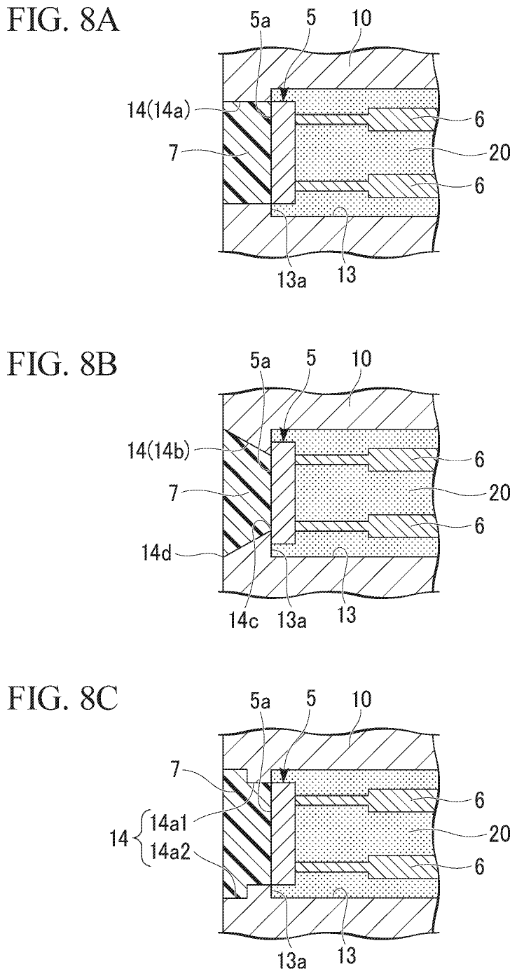

For example, the shape of the light projection aperture 14 may be changed, and the shapes shown in FIGS. 8A to 8C may be adopted.

In FIG. 8A, the light projection aperture 14 does not have the tapered part 14b and is constituted by only the straight part 14a. Additionally, the front opening edge and the rear opening edge of the light projection aperture 14 have the same shape. In this case, the processing of the case 10 becomes easier.

In FIG. 8B, the light projection aperture 14 does not have the straight part 14a and is constituted by only the tapered part 14b. Additionally, the tapered part 14b is formed from the rear opening edge 14c of the light projection aperture 14 to the front opening edge 14d thereof. In this case, the light emitted from the light emitting element 5 is more directly emitted forward along the tapered part 14b. Therefore, it is possible to more efficiently illuminate the image capturing target. Moreover, the opening area A2 of the front end of the light projection aperture 14 can be made larger than the area A1 of the light emitting surface 5a.

In FIG. 8C, the light projection aperture 14 does not have the tapered part 14b and has two straight parts 14a1 and 14a2 having different cross-sectional areas. The second straight part 14a2 is located in front of the first straight part 14a1 and opens to the front surface 10a of the case 10. Additionally, the cross-sectional area of the second straight part 14a2 is larger than the cross-sectional area of the first straight part 14a1. According to this shape, the processing of the case 10 can be facilitated more easily than in a case where the tapered part 14b is formed, and the opening area A2 in front of the light projection aperture 14 can be made larger than the area A1 of the light emitting surface 5a.

Additionally, although the rear opening edge 14c (see FIG. 4) of the light projection aperture 14 in the above-described embodiments has an oblong shape with curved corners, the shape of the rear opening edge may be changed. For example, when the light emitting surface 5a of the light emitting element 5 has a square shape, the rear opening edge 14c may have a substantially square shape obtained by curving the corners in the shape of the light emitting surface 5a. Similarly, when the light emitting surface 5a has an N-angle shape (polygonal shape), the rear opening edge 14c may have a shape obtained by curving the corners of the N-angle shape. Also in these cases, most other than the angular parts 5c can be caused to face the light projection aperture 14 while the angular parts 5c of the light emitting surface 5a is butted against the abutting surface 13a. Therefore, it is possible to more efficiently radiate the light emitted by the light emitting element 5 forward through the recess while positioning the light emitting element 5.

Additionally, the rear opening edge 14c of the light projection aperture 14 may not have a curved part. If the area inside the rear opening edge 14c (the opening area behind the light projection aperture 14) is smaller than the area of the light emitting surface 5a of the light emitting element 5, it is possible to butt at least the light emitting surface 5a against the abutting surface 13a to position the light emitting element 5.

In addition, it is possible to appropriately replace the constituent elements in the above-described embodiments with well-known constituent elements without departing from the spirit of the present invention, and the above-described embodiments and modified examples may be appropriately combined.

### INDUSTRIAL APPLICABILITY

According to one or more embodiments of the present invention, it is possible to provide an image capturing module capable of preventing a light emitting element from falling off.

Furthermore, although the disclosure has been described with respect to only a limited number of embodiments, those skilled in the art, having benefit of this disclosure, will appreciate that various other embodiments may be devised without departing from the scope of the present invention. Accordingly, the scope of the invention should be limited only by the attached claims.

### REFERENCE SIGNS LIST

1 Endoscope

2 Image capturing module

4 Image capturing element

5 Light emitting element

5a Light emitting surface

7 Transparent resin

10 Case

13 Light housing part

13a abutting surface

14 Light projection aperture

14b Tapered part

14c Rear opening edge

1. An image capturing module comprising:
an image capturing element;
a light emitting element; and
a case that is configured to hold the image capturing element and the light emitting element, wherein the case has:
a light projection aperture that is configured to open to a front surface of the case; and
a light housing part disposed behind the light projection aperture and houses the light emitting element,
the light housing part is connected to the light projection aperture inside the case, and a rear end of the light projection aperture opens to a light emitting surface of the light emitting element, and
an opening area of the rear end of the light projection aperture is smaller than an area of the light emitting surface of the light emitting element.

2. The image capturing module according to claim 1, wherein the light projection aperture has a tapered part that is inclined, and a cross-sectional area of the light projection aperture increases toward a front opening edge of the light projection aperture.

3. The image capturing module according to claim 2, wherein the tapered part is formed from a rear opening edge of the light projection aperture to the front opening edge thereof.

4. The image capturing module according to claim 1, wherein a transparent resin is filled inside the light projection aperture.

5. The image capturing module according to claim 4, wherein the transparent resin contains light diffusing particles.

6. The image capturing module according to claim 1, wherein an area A1 of the light emitting surface and an opening area A2 of a front end of the light projection aperture satisfy A1<A2.

7. An endoscope using the image capturing module according to claim 1.
